(19) United States
(12) Patent Application Publication
(10) Pub. No.: US 2026/0324722 A1
(43) Pub. Date: Oct. 22, 2026

(54) SMOKING SUBSTITUTE APPARATUS

(71) Applicant: Imperial Tobacco Limited, Bristol (GB)

(72) Inventors: Samantha Murray, Bristol (GB); Chris Lord, Bristol (GB); David Jones, Bristol (GB); Jonathan Marchbank, Bristol (GB); Tom Sudlow, Bristol (GB); Ian Stuart, Bristol (GB)

(21) Appl. No.: 19/532,457

(22) Filed: Feb. 6, 2026

Related U.S. Application Data
(63) Continuation of application No. 17/395,276, filed on Aug. 5, 2021, now abandoned.
Application No. 17/395,276 is a continuation of application No. PCT/EP2020/053106, filed on Feb. 7, 2020.
Application No. PCT/EP2020/053106 is a continuation of application No. PCT/EP2020/053110, filed on Feb. 7, 2020.
Application No. PCT/EP2020/053110 is a continuation of application No. PCT/EP2020/053112, filed on Feb. 7, 2020.
Application No. PCT/EP2020/053112 is a continuation of application No. PCT/EP2020/053115, filed on Feb. 7, 2020.
Application No. PCT/EP2020/053115 is a continuation of application No. PCT/EP2020/053116, filed on Feb. 7, 2020.
Application No. PCT/EP2020/053116 is a continuation of application No. PCT/EP2020/053119, filed on Feb. 7, 2020.
Application No. PCT/EP2020/053119 is a continuation of application No. PCT/EP2020/053120, filed on Feb. 7, 2020.
Application No. PCT/EP2020/053120 is a continuation of application No. PCT/EP2020/053130, filed on Feb. 7, 2020.
Application No. PCT/EP2020/053130 is a continuation of application No. PCT/EP2020/053139, filed on Feb. 7, 2020.
Application No. PCT/EP2020/053139 is a continuation of application No. PCT/EP2020/053141, filed on Feb. 7, 2020.
Application No. PCT/EP2020/053141 is a continuation of application No. PCT/EP2020/053176, filed on Feb. 7, 2020.
Application No. PCT/EP2020/053176 is a continuation of application No. PCT/EP2020/053186, filed on Feb. 7, 2020.
Application No. PCT/EP2020/053186 is a continuation of application No. PCT/EP2020/053189, filed on Feb. 7, 2020.
Application No. PCT/EP2020/053189 is a continuation of application No. PCT/EP2020/053190, filed on Feb. 7, 2020.
Application No. PCT/EP2020/053190 is a continuation of application No. PCT/EP2020/053191, filed on Feb. 7, 2020.
Application No. PCT/EP2020/053191 is a continuation of application No. PCT/EP2020/053193, filed on Feb. 7, 2020.
Application No. PCT/EP2020/053193 is a continuation of application No. PCT/EP2020/053194, filed on Feb. 7, 2020.
Application No. PCT/EP2020/053194 is a continuation of application No. PCT/EP2020/053195, filed on Feb. 7, 2020.
Application No. PCT/EP2020/053195 is a continuation of application No. PCT/EP2020/053200, filed on Feb. 7, 2020.

(30) Foreign Application Priority Data
Feb. 7, 2019 (EP) 19155858.4
Feb. 7, 2019 (EP) 19155861.8
Feb. 7, 2019 (EP) 19155862.6
Feb. 7, 2019 (EP) 19155864.2
Feb. 7, 2019 (EP) 19155866.7
Feb. 7, 2019 (EP) 19155871.7
Feb. 7, 2019 (EP) 19155873.3
Feb. 7, 2019 (EP) 19155875.8
Feb. 7, 2019 (EP) 19155877.4
Feb. 7, 2019 (EP) 19155878.2
Feb. 7, 2019 (EP) 19155879.0
Feb. 7, 2019 (EP) 19155887.3
Feb. 7, 2019 (EP) 19155894.9
Feb. 7, 2019 (EP) 19155903.8
Feb. 7, 2019 (EP) 19155908.7
Feb. 7, 2019 (EP) 19155929.3
Feb. 7, 2019 (EP) 19155933.5
Feb. 7, 2019 (EP) 19155934.3
Feb. 7, 2019 (EP) 19155936.8
Feb. 7, 2019 (EP) 19155945.9

(51) Int. Cl. A24F 40/42; A24B 15/167; A24B 15/28; A24F 40/10; A24F 40/485; A24F 40/70; A61M 15/00

(52) U.S. Cl. CPC A24F 40/42; A24B 15/167; A24B 15/285; A24F 40/10; A24F 40/485; A24F 40/70; A61M 15/009; A61M 2205/0238

(57) ABSTRACT

Disclosed is a smoking substitute apparatus comprising a first passage for receipt of an aerosol flow, a second passage arranged in parallel with the first passage and comprising an additive delivery portion for delivering an additive to an airflow in the second passage, a mouthpiece comprising an outlet in fluid communication with the first and second passages, and a valve for controlling aerosol flow through the first passage. The valve is movable between a first position in which an airflow drawn through the outlet is received substantially exclusively from the first passage, and a second position in which an airflow drawn through the outlet is received from a combination of the first and second passages.

### CROSS REFERENCE TO RELATED APPLICATIONS/INCORPORATION BY REFERENCE STATEMENT

This application is a continuation of United States nonprovisional utility patent application no. Ser. No. 17/395,276, filed on 5 Aug. 2021 and published on 27 Jan. 2022 as United States patent application publication no. US 2022/0022537A 1 , which is a continuation of the following international patent applications: no. PCT/EP 2020/053119, filed on 7 Feb. 2020, which claims priority to EP 19155894.9, filed on 7 Feb. 2019; no. PCT/EP 2020/053193, filed on 7 Feb. 2020, which claims priority to EP 19155934.3, filed on 7 Feb. 2019; no. PCT/EP 2020/053195, filed on 7 Feb. 2020, which claims priority to EP 19155936.8, filed on 7 Feb. 2019; no. PCT/EP 2020/053112, filed on 7 Feb. 2020, which claims priority to EP 19155866.7, filed on 7 Feb. 2019; no. PCT/EP 2020/053186, filed on 7 Feb. 2020, which claims priority to EP 19155903.8, filed on 7 Feb. 2019; no. PCT/EP 2020/053116, filed on 7 Feb. 2020, which claims priority to EP 19155871.7, filed on 7 Feb. 2019; no. PCT/EP 2020/053194, filed on 7 Feb. 2020, which claims priority to EP 19155945.9, filed on 7 Feb. 2019; no. PCT/EP 2020/053189, filed on 7 Feb. 2020, which claims priority to EP 19155908.7, filed on 7 Feb. 2019; no. PCT/EP 2020/053120, filed on 7 Feb. 2020, which claims priority to EP 19155873.3, filed on 7 Feb. 2019; no. PCT/EP 2020/053115, filed on 7 Feb. 2020, which claims priority to EP 19155861.8, filed on 7 Feb. 2019; no. PCT/EP 2020/053130, filed on 7 Feb. 2020, which claims priority to EP 19155875.8, filed on 7 Feb. 2019; no. PCT/EP 2020/053139, filed on 7 Feb. 2020, which claims priority to EP 19155877.4, filed on 7 Feb. 2019; no. PCT/EP 2020/053141, filed on 7 Feb. 2020, which claims priority to EP 19155878.2, filed on 7 Feb. 2019; no. PCT/EP 2020/053191, filed on 7 Feb. 2020, which claims priority to EP 19155929.3, filed on 7 Feb. 2019; no. PCT/EP 2020/053110, filed on 7 Feb. 2020, which claims priority to EP 19155862.6, filed on 7 Feb. 2019; no. PCT/EP 2020/053106, filed on 7 Feb. 2020, which claims priority to EP 19155864.2, filed on 7 Feb. 2019; no. PCT/EP 2020/053190, filed on 7 Feb. 2020, which claims priority to EP 19155887.3, filed on 7 Feb. 2019; no. PCT/EP 2020/053176, filed on 7 Feb. 2020, which claims priority to EP 19155879.0, filed on 7 Feb. 2019; and no. PCT/EP 2020/053200, filed on 7 Feb. 2020, which claims priority to EP 19155858.4, filed on 7 Feb. 2019, and to EP 19155933.5, filed on 7 Feb. 2019. The entire contents of each of the above-referenced applications and the above-referenced published patent application are hereby incorporated herein by reference in their entirety.

### FIELD OF THE INVENTION

The present invention relates to a smoking substitute apparatus and, in particular, a smoking substitute apparatus that is able to deliver an additive (e.g. a flavourant) to a user.

### BACKGROUND

The smoking of tobacco is generally considered to expose a smoker to potentially harmful substances. It is generally thought that a significant amount of the potentially harmful substances is generated through the heat caused by the burning and/or combustion of the tobacco and the constituents of the burnt tobacco in the tobacco smoke itself.

Combustion of organic material such as tobacco is known to produce tar and other potentially harmful by-products. There have been proposed various smoking substitute systems in order to avoid the smoking of tobacco.

Such smoking substitute systems can form part of nicotine replacement therapies aimed at people who wish to stop smoking and overcome a dependence on nicotine.

Smoking substitute systems include electronic systems that permit a user to simulate the act of smoking by producing an aerosol (also referred to as a “vapor”) that is drawn into the lungs through the mouth (inhaled) and then exhaled. The inhaled aerosol typically bears nicotine and/or a flavorant without, or with fewer of, the odor and health risks associated with traditional smoking.

In general, smoking substitute systems are intended to provide a substitute for the rituals of smoking, whilst providing the user with a similar experience and satisfaction to those experienced with traditional smoking and with combustible tobacco products.

The popularity and use of smoking substitute systems has grown rapidly in the past few years. Although originally marketed as an aid to assist habitual smokers wishing to quit tobacco smoking, consumers are increasingly viewing smoking substitute systems as desirable lifestyle accessories. There are a number of different categories of smoking substitute systems, each utilizing a different smoking substitute approach.

One approach is the so-called “vaping” approach, in which a vaporizable liquid, typically referred to (and referred to herein) as “e-liquid”, is heated by a heating device (referred to herein as an electronic cigarette or “e-cigarette” device) to produce an aerosol vapor which is inhaled by a user. The e-liquid typically includes a base liquid as well as nicotine and/or a flavorant. The resulting vapor therefore also typically contains nicotine and/or a flavorant. The base liquid may include propylene glycol and/or vegetable glycerin.

A typical e-cigarette device includes a mouthpiece, a power source (typically a battery), a tank for containing e-liquid, as well as a heating device. In use, electrical energy is supplied from the power source to the heating device, which heats the e-liquid to produce an aerosol (or “vapor”) which is inhaled by a user through the mouthpiece.

E-cigarettes can be configured in a variety of ways. For example, there are “closed system” vaping smoking substitute systems, which typically have a sealed tank and heating element. The tank is pre-filled with e-liquid and is not intended to be refilled by an end user. One subset of closed system vaping smoking substitute systems includes a main body which includes the power source, wherein the main body is configured to be physically and electrically coupled to a consumable including the tank and the heating element. In this way, when the tank of a consumable has been emptied, that consumable is disposed of. The main body can be reused by connecting it to a new, replacement, consumable. Another subset of closed system vaping smoking substitute systems is completely disposable, and intended for one-use only.

There are also “open system” vaping smoking substitute systems which typically have a tank that is configured to be refilled by a user. In this way the entire device can be used multiple times.

An example vaping smoking substitute system is the myblu™ e-cigarette. The myblu™ e-cigarette is a closed system which includes a main body and a consumable. The main body and consumable are physically and electrically coupled together by pushing the consumable into the main body. The main body includes a rechargeable battery. The consumable includes a mouthpiece, a sealed tank which contains e-liquid, as well as a heater, which for this device is a heating filament coiled around a portion of a wick. The wick is partially immersed in the e-liquid, and conveys e-liquid from the tank to the heating filament. The device is activated when a microprocessor on board the main body detects a user inhaling through the mouthpiece. When the device is activated, electrical energy is supplied from the power source to the heating device, which heats e-liquid from the tank to produce a vapor which is inhaled by a user through the mouthpiece.

For a smoking substitute device, it is desirable to deliver nicotine into the user's lungs, where it can be absorbed into the bloodstream. As explained above, in the so-called “vaping” approach, e-liquid is heated by a heating device to produce an aerosol vapor which is inhaled by a user. Many e-cigarettes also deliver flavor to the user to enhance the experience. In such e-cigarettes, flavor compounds are contained in the e-liquid that is heated. However, in some cases, toxicology restrictions are placed on the amount of flavor that can be contained in the e-liquid, and this can result in some e-liquid flavors delivering a weak and underwhelming taste sensation to consumers in the pursuit of safety. Further, there is a view that providing a flavourant as part of the e-liquid, such that the flavourant is vaporised with the e-liquid, may be disadvantageous.

There may be a need for improved design of smoking substitute systems, in particular in regards to the delivery of flavor to a user.

The present disclosure has been devised in the light of the above considerations.

### SUMMARY OF THE INVENTION

At its most general, the present invention relates to a smoking substitute apparatus comprising a user controllable valve allowing a user to select a proportion of additive received through a mouthpiece of the apparatus.

According to a first aspect there is provided a smoking substitute apparatus comprising: a first passage for receipt of an aerosol flow; a second passage arranged in parallel with the first passage and comprising an additive delivery portion for delivering an additive to an airflow in the second passage; a mouthpiece comprising an outlet in fluid communication with the first and second passages; and a valve for controlling aerosol flow through the first passage, the valve movable between: a first position in which an airflow drawn through the outlet is received substantially exclusively from first passage; and a second position in which an airflow drawn through the outlet is received from a combination of the first and second passages.

The smoking substitute apparatus may provide a convenient means for providing an additive separately to an aerosol (e.g., where the second passage is for receipt of an aerosol). Providing an additive that is separate to the e-liquid may provide more versatility in how flavor can be delivered to the user. The user controllable valve allows a user to control the amount of additive provided to the user via the outlet.

The phrase “substantially exclusively” means that minimal airflow is received (or no airflow is received) at the outlet from the second passage when the valve is in the first position. For example, if any airflow is received from the second passage, that airflow may only be incidental and may not contribute in any significant way to the characteristics of the airflow received at the outlet. For example, where the additive delivery portion delivers a flavorant to the airflow, that flavorant may not be detectable by a user when the valve is in the first position.

For example, when the valve is in the first position, the proportion of the airflow received at the outlet that is received from the first passage may be greater than 90%. The proportion of the airflow received at the outlet (when the valve is in the open position) that is received from the first passage may be greater than 95%. The proportion of the airflow received at the outlet that is received from the first passage may be 100%.

In the first position, the first passage may be substantially unobstructed by the valve. Thus, the valve may be in a retracted position.

In the first position the second passage may be substantially unobstructed by the valve. In the second position the second passage may be substantially unobstructed by the valve. In that respect, both the first and second passages may be considered open (i.e., to airflow) when the valve is in the first position. The second passage may have a greater resistance (or substantially greater resistance) to airflow than the first passage. In this way, a path of least resistance may be defined through the first passage. Thus, when the valve is in the first position (i.e., and both passages are open) air may only flow through the first passage and not the second passage due to the second passage having the greater resistance to airflow.

In the second position the first passage may at least partially obstructed by the valve. This obstruction may provide some resistance to airflow in the first passage. In other words, the resistance to airflow of the first passage may be greater when the valve is in the second position compared to when the valve is in the first position. This may mean that the resistance to airflow of the first and second passages are equal (or similar) such that a combination of airflows from the first and second passages is received when air is drawn from the outlet. That is, airflow through the second passage may indirectly controlled by movement of the valve of the first passage (i.e., due to that movement altering a resistance to airflow of the first passage).

The valve may be movable to a third position in which an airflow drawn through the outlet is received substantially exclusively from the second passage. In the third position, the valve substantially obstructs the first passage. This obstruction of the first passage may prevent (or substantially prevent) airflow from being drawn through the first passage.

Thus, a single valve may provide an airflow drawn through the outlet that is exclusively from the first passage, exclusively from the second passage, or from a combination of the first and second passages.

The second passage may comprise an airflow restrictor for restricting airflow through the second passage. The airflow restrictor may, for example, be in the form of a narrower section in the passage.

Alternatively, the airflow restrictor may comprise a channel that follows a tortuous path.

The additive delivery portion may be configured to restrict airflow through the second passage. The additive delivery portion may comprise a porous medium (e.g., a porous element). The porous medium may provide partial obstruction of the second passage so as to restrict airflow through the second passage. An additive may be deposited on the porous medium. Alternatively, the porous medium may be impregnated with the additive.

The additive delivery portion may be in the form of a porous wick. The porous wick may extend across at least a portion of the passage. A portion of the porous wick may extend into an additive reservoir (e.g., a tank containing an additive). In this way, the additive may be conveyed from the reservoir along the wick (i.e., so as to be exposed to an airflow in the passage).

The additive delivery portion may alternatively be in the form of a portion of the passage (e.g., of a wall of the passage) comprising an additive coating.

The additive delivery portion may be in the form of a plurality of particles packed in a container (e.g., a mesh container). In this respect, air may flow through gaps formed between the packed particles.

The valve may be biased into the first position. The valve may be biased by a spring. For example, the valve may be biased by a compression spring or a torsion spring.

Movement of the valve between the first and second positions may be in a direction generally transverse to a longitudinal axis of the first passage. Movement of the valve between the first and second positions may be substantially perpendicular to the longitudinal axis of the first passage. The valve may be slidably engaged with a wall of the passage (and/or a wall of a housing of the apparatus).

The valve may comprise a pushbutton for a user to move the valve between the first and second positions. The valve may comprise a barrier for obstructing the first passage. The barrier may be rigidly connected to the pushbutton. The pushbutton may define a first end of the valve and the barrier may define an opposing end of the valve. When the valve is in the first position the pushbutton may project beyond an outer surface of a housing of the apparatus. A compression spring may be disposed between the barrier and an opposing wall of the first passage so as to bias the valve into the first position (e.g., such that the barrier is retracted from the first passage). A sealing portion may be disposed between the valve and a wall of the passage so as to prevent leakage of an airflow (e.g., including an aerosol) from the first passage.

The valve may be configured to rotate between the first and second positions. The valve may hinge between the first and second positions. In that respect, the valve may be hingably mounted (e.g., to a wall of the first passage). In this respect, the valve may be in the form of a flap.

The valve may be configured to move from the first position to the second position in response to an airflow drawn from the outlet. The valve may be configured to move from the first position to the second position in response to a pressure drop in the first passage. The pressure drop may be a predetermined pressure drop. That is, the valve may only move from the first position to the second position in response to a predetermined pressure in the first passage. The predetermined pressure may be a pressure indicative of a user inhaling at the outlet. The valve may be configured to move in response to the magnitude of an inhale (e.g., flow rate drawn from outlet) by a user at the outlet. In this way, a proportion of airflow from the second passage (received through the outlet) may be controlled by the strength of an inhalation at the outlet. Thus, for example, where the valve is a flap, the valve may rotate (e.g., about a hinge) in the first passage driven by an airflow drawn from the outlet.

Alternatively, the valve may comprise an actuator for moving the valve. The actuator portion may be movable in response to a signal received from, e.g., a controller. The controller may control the actuator in response to a signal received from a sensor. For example, the apparatus may comprise a puff sensor for detecting inhalation at the outlet and the controller may be configured to control the actuator in response to a signal indicative of inhalation at the outlet. The apparatus may comprise, e.g., a pressure sensor for detecting a pressure in the first or second passage. The controller may control the actuator in response to a signal indicative of a pressure in the first or second passage. Thus, for example, the controller may move the valve from the first position to the second position in response to a pressure drop.

The additive delivery portion may be configured to deliver a flavorant to an airflow in the second passage. In that respect, the additive delivery portion may comprise a flavorant. The term “flavorant” may describe a compound or combination of compounds that provide flavor and/or aroma. For example, the flavorant may be configured to interact with a sensory receptor of a user (such as an olfactory or taste receptor). The flavorant may include one or more volatile substances.

The flavorant may be provided in solid or liquid form. The flavorant may be natural or synthetic. For example, the flavorant may include menthol, licorice, chocolate, fruit flavor (including, e.g., citrus, cherry etc.), vanilla, spice (e.g., ginger, cinnamon) and tobacco flavor. The flavorant may be evenly dispersed or may be provided in isolated locations and/or varying concentrations.

The smoking substitute apparatus may be in the form of a consumable. The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable.

Alternatively, the smoking substitute apparatus may be a non-consumable apparatus (e.g., that is in the form of an open smoking substitute system). In such embodiments an aerosol former (e.g., e-liquid) of the system may be replenished by re-filling, e.g., a reservoir of the smoking substitute apparatus with the aerosol former (rather than replacing a consumable component of the apparatus).

In light of this, it should be appreciated that some of the features described herein as being part of the smoking substitute apparatus may alternatively form part of a main body for engagement with the smoking substitute apparatus (i.e., when the smoking substitute apparatus is in the form of a consumable).

Where the smoking substitute apparatus is in the form of a consumable, the main body and the consumable may be configured to be physically coupled together. For example, the consumable may be at least partially received in a recess of the main body, such that there is an interference fit between the main body and the consumable. Alternatively, the main body and the consumable may be physically coupled together by screwing one onto the other, or through a bayonet fitting.

Thus, the smoking substitute apparatus may comprise one or more engagement portions for engaging with a main body. In this way, one end of the smoking substitute apparatus may be coupled with the main body, whilst an opposing end of the smoking substitute apparatus may define a mouthpiece of the smoking substitute system.

The smoking substitute apparatus may comprise a reservoir configured to store an aerosol former, such as an e-liquid. The e-liquid may, for example, comprise a base liquid and, e.g., nicotine. The base liquid may include propylene glycol and/or vegetable glycerin. The e-liquid may be flavorless. That is, the e-liquid may not contain any flavorants and may consist solely of a base liquid of propylene glycol and/or vegetable glycerin and nicotine.

The reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The reservoir may be referred to as a “clearomizer” if it includes a window, or a “cartomizer” if it does not. The first passage may be at least partially defined by the tank. The tank may substantially (or fully) define the first passage. In this respect, the tank may surround the first passage.

The first passage may comprise an aerosol generator for delivering an aerosol to an airflow in the first passage. The valve may be between the aerosol generator and the outlet. That is, the aerosol generator may be upstream of the valve. The aerosol generator may comprise a wick. The aerosol generator may further comprise a heater. The wick may comprise a porous material. A portion of the wick may be exposed to airflow in the first passage. The wick may also comprise one or more portions in contact with liquid stored in the reservoir. For example, opposing ends of the wick may protrude into the reservoir and a central portion (between the ends) may extend across the passage so as to be exposed to air flow in the passage. Thus, fluid may be drawn (e.g., by capillary action) along the wick, from the reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in air flowing through the first passage. This vapor may subsequently cool to form an aerosol in the first passage.

The first and second passages may each comprise a respective inlet. When the apparatus is in the form of a consumable, each inlet may be formed in an end of the consumable for engagement with main body. Alternatively, the first and second passages may share an inlet. A mixing chamber may be fluidly connected between the passages and the outlet. When the valve is in the second position, the airflows from the first and second passages may mix in the mixing chamber prior to being drawn through the outlet. Alternatively, the first and second passages may extend to the outlet (e.g., such that mixing instead occurs in a user's mouth once inhaled).

The passages may be generally parallel to one another. The passages may be separated by a separator in the form of a wall extending between the passages. Each of the passages may have a generally continuous (i.e., constant cross-section) along its length.

The smoking substitute apparatus (or main body engaged with the smoking substitute apparatus) may comprise a power source. The power source may be electrically connected (or connectable) to a heater of the smoking substitute apparatus (e.g., when engaged with the main body). The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable.

The electrical interface may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute apparatus or main body may comprise a controller, which may include a microprocessor. The controller may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method. As is provided above, the controller may be configured to control the valve (e.g., an actuator for moving the valve). Thus, the controller may control the valve to move between the first and second positions.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

As is provided above, a puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. When the smoking substitute apparatus is in the form of a consumable, the puff sensor may form part of the consumable or the main body.

The invention includes the combination of the aspects and preferred features described except where such a combination is clearly impermissible or expressly avoided.

### SUMMARY OF THE FIGURES

So that the invention may be understood, and so that further aspects and features thereof may be appreciated, embodiments illustrating the principles of the invention will now be discussed in further detail with reference to the accompanying figures, in which:

FIG. 1A is a front view of a smoking substitute system, according to a first embodiment, in an engaged position.

FIG. 1B is a front view of smoking substitute system of the first embodiment in a disengaged position.

FIG. 1C is a section view of an aerosol delivery device of the first embodiment in a first position.

FIG. 1D is a section view of an aerosol delivery device of the first embodiment in a second position.

FIG. 1E is a section view of an aerosol delivery device of the first embodiment in a third position.

FIGS. 2A and 2B are schematics depicting a valve according to a second embodiment.

FIGS. 3A and 3B are schematics depicting a valve according to a third embodiment.

FIGS. 4A and 4B are schematics depicting a valve according a fourth embodiment.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

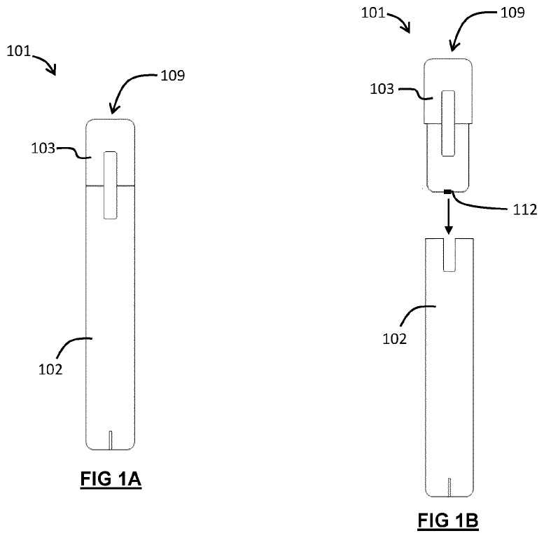

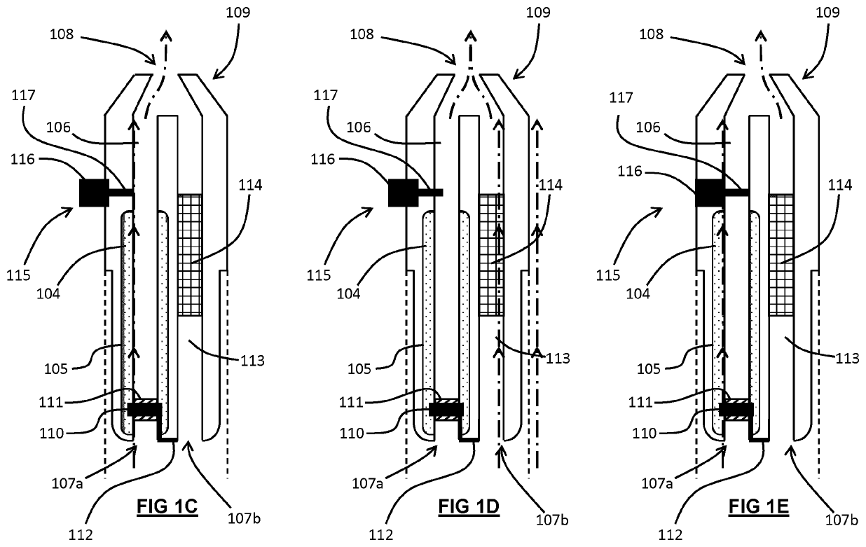

FIGS. 1A and 1B illustrate a smoking substitute system in the form of an e-cigarette system 101. The system 101 comprises an e-cigarette device defining a main body 102 of the system 101, and an aerosol delivery device in the form of an e-cigarette consumable (or “pod”) 103. In the illustrated embodiment the consumable 103 (aerosol delivery device) is removable from the main body (e-cigarette device), so as to be a replaceable component of the system 101. In other words, the e-cigarette system 101 is a closed system.

As is apparent from FIGS. 1A and 1B, the consumable 103 is configured to engage the main body 102. FIG. 1A shows the main body 102 and the consumable 103 in an engaged state, whilst FIG. 1B shows the main body 102 and the consumable 103 in a disengaged state. When engaged, a portion of the consumable 103 is received in a cavity of the main body 102 and is retained in the engaged position by way of a snap-engagement mechanism. In other embodiments, the main body 102 and consumable 103 may be engaged by screwing one into (or onto) the other, through a bayonet fitting, or by way of an interference fit.

The system 101 is configured to vaporize an aerosol-former, which in the illustrated embodiment, is in the form of a nicotine-based e-liquid 104. The e-liquid 104 comprises nicotine and a base liquid including propylene glycol and/or vegetable glycerin. In the present embodiment, the e-liquid 104 is flavorless (and does not include any added flavorant). That is, if the e-liquid 104 were to be inhaled (i.e., in aerosol form) by a user, it would not have a particularly perceptible flavor or taste.

As is more apparent from FIGS. 1C to 1E, this e-liquid 104 is stored within a reservoir in the form of a tank 105 that forms part of the consumable 103. In the illustrated embodiment, the consumable 103 is a “single-use” consumable 103. That is, upon exhausting the e-liquid 104 in the tank 105, the intention is that the user disposes of the entire consumable 103. In other embodiments, the e-liquid (i.e., aerosol former) may be the only part of the system that is truly “single-use”. That is, the tank may be refillable with e-liquid or the e-liquid may be stored in a non-consumable component of the system. For example, the e-liquid may be stored in a tank located in the main body or stored in another component that is itself not single-use (e.g., a refillable cartomizer).

The tank 105 surrounds, and thus defines a portion of, a first passage 106 that extends between an inlet 107a and an outlet 108 at opposing ends of the consumable 103. In this respect, the passage 106 comprises an upstream end at the end of the consumable 103 that engages with the main body 102, and a downstream end at an opposing end of the consumable 103 that comprises a mouthpiece 109 of the system 101. When the consumable 103 is engaged with the main body 102, a user can inhale (i.e., take a puff) via the mouthpiece 109 so as to draw air through the passage 106, and so as to form an airflow (indicated by arrows in FIGS. 1C and 1E) in a direction from the inlet 107a to the outlet 108. The first passage 106 is in fluid communication with a gap defined between the consumable 103 and the main body 102 (when engaged) such that air outside of the system 101 is drawn into the first passage 106 (during an inhale).

The smoking substitute system 101 is configured to vaporize the e-liquid 104 for inhalation by a user. To provide this, the consumable 103 comprises a heater having a porous wick 110 and a resistive heating element in the form of a heating filament 111 that is helically wound around a portion of the porous wick 110. The porous wick 110 extends across the first passage 106 (i.e., transverse to a longitudinal axis of the passage 106) and opposing ends of the wick 110 extend into the tank 105 (so as to be submerged in the e-liquid 104). In this way, e-liquid 104 contained in the tank 105 is conveyed from the opposing ends of the porous wick 110 to a central portion of the porous wick 110 so as to be exposed to the airflow in the first passage 106 (i.e., caused by a user inhaling). In other embodiments the heating filament 111 and/or wick 110 may form part of the main body (but may engage the tank 105 during engagement of the main body 102 and the consumable 103).

The helical filament 111 is wound about this exposed central portion of the porous wick 110 and is electrically connected to an electrical interface in the form of electrical contacts 112 mounted at the end of the consumable that is proximate the main body 102 (when engaged). When the consumable 103 is engaged with the main body 102, the electrical contacts 112 contact corresponding electrical contacts (not shown) of the main body 102. The main body electrical contacts are electrically connected to a power source (not shown) of the main body 102, such that (in the engaged position) the filament 111 is electrically connected to the power source. In this way, power can be supplied by the main body 102 to the filament 111 in order to heat the filament 111. This heat is transferred from the filament 111 to the porous wick 110 which causes e-liquid 104 conveyed by the porous wick 110 to increase in temperature to a point at which it vaporizes. The vaporized e-liquid becomes entrained in the airflow in the first passage 106 and, between the vaporization point at the filament 111 and the outlet 108, condenses to form an aerosol. This aerosol is then inhaled, via the mouthpiece 109, by a user of the system 101.

The power source of the main body 102 may be in the form of a battery (e.g., a rechargeable battery).

The main body 102 may comprise a connector in the form of, e.g., a USB port for recharging this battery. The main body 102 may also comprise a controller that controls the supply of power from the power source to the main body electrical contacts (and thus to the filament 111). That, is the controller may be configured to control a voltage applied across the main body electrical contacts, and thus the voltage applied across the filament 111. In this way, the filament 111 may only be heated under certain conditions (e.g., during a puff and/or only when the system is in an active state). In this respect, the main body 102 may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102 and consumable 103 may comprise a further interface which may, for example, be in the form of an RFID reader, a barcode or QR code reader. This interface may be able to identify a characteristic (e.g., a type) of a consumable 103 engaged with the main body 102. In this respect, the consumable 103 may include any one or more of an RFID chip, a barcode or QR code, or memory within which is an identifier and which can be interrogated via the interface.

The consumable 103 additionally comprises a second passage 113 that is arranged in parallel with the first passage 106 and (like the first passage 106) is in fluid communication with the outlet 108. Thus, the second passage 113 extends between an inlet 107b-2 and the outlet 108. The second passage 113 comprises an additive delivery portion, in the form of a porous element 114 impregnated with a flavorant. The porous element 114 is located in the second passage 113 between the inlet 107b-2 and the outlet 108 so as to partially obstruct the passage 113. This porous element 114 delivers a flavorant to an airflow as it flows through the filter 114. Thus, when air is drawn through the outlet 108 and flows from the second passage 113 flavorant is carried from the porous element 114 to the outlet 108.

Because the porous element 114 partially obstructs the passage 113, In addition to delivering a flavorant, the porous element 114 acts as an airflow restrictor. In other words, the porous element 114 increases the resistance to airflow of the second passage 113, such that the resistance to airflow of the second passage 113 is substantially greater than the resistance to airflow of the first passage 106. As will be described further below, this allows the airflow through the second passage 113 to be controlled by way of a valve 115 forming part of the first passage 106.

The valve 115 is movable transversely across the first passage 106 between first (FIG. 1C), second (FIG. 1D) and third (FIG. 1E) positions. That is, the valve 115 is movable progressively from a position in which the first passage 106 is unobstructed by the valve (i.e., the first position) to a position in which is obstructs the first passage 106 (i.e., the third position). In the illustrated embodiment, the valve is slidably engaged with a wall of the first passage 106 and comprises a barrier 117] and a pushbutton 116 rigidly connected to the barrier 117. Thus, a user can press the pushbutton 116 to move the barrier 117 transversely across the first passage 106.

In the first position (FIG. 1C) the barrier 117 is retracted so as not to extend into the first passage 106. In this position, the second passage 113 has a substantially greater resistance to airflow than the first passage 106. Hence, the first passage 106 defines a path of least resistance an airflow drawn through the outlet 108 (as shown by the arrows) will flow through the first passage 106 and not the second passage 113. That is, the airflow received at the outlet 108 is exclusively formed of aerosol flowing from the first passage 106.

In the second position (FIG. 1D, the barrier 117 extends partway across the first passage 106. In this position the barrier 117 increases the resistance to airflow of the first passage 106 such that air (drawn through the outlet 108) flows through both the first 106 and second 113 passages. Thus, the airflow received at the outlet 108 is a combination of aerosol (flowing through the first passage 106) and flavored air (flowing from the second passage 113).

In the third position (FIG. 1E), the barrier 117 extends across the first passage 106 so as to fully obstruct the first passage 106. Thus, air is forced to flow through the second passage 113 and the air received at the outlet 108 is exclusively formed of flavored air received from the second passage 113.

As should be apparent, the valve 115 allows a user to control (or select) the amount of additive (in this case flavorant) received through the outlet 108.

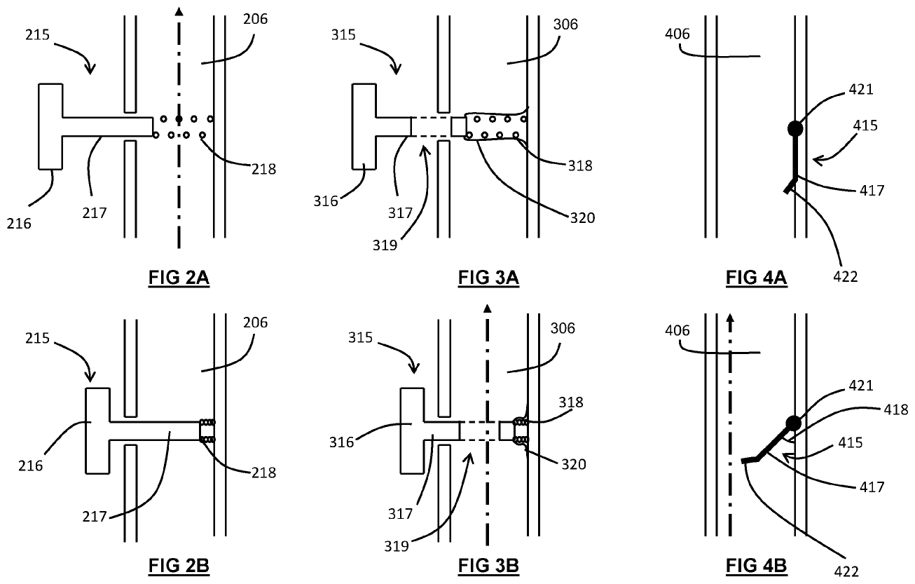

FIGS. 2A to 4B illustrate three exemplary valves that may, for example, form part of the substitute smoking system described above with reference to FIGS. 1A to 1E.

In FIGS. 2A and 2B, the valve 215 is similar to that described above, except that it further comprises a biasing means in the form of a compression spring 218. The compression spring 218 is disposed between a distal end of the barrier 217 and an opposing wall of the first passage 206, and is configured to urge the valve 215 towards the first position (i.e., retracted from the first passage 206). Thus, in order to move the valve 215 from the first position (FIG. 2A) to the third position (FIG. 2B), a user must push the valve 215 against the biasing force of the spring 218.

The valve 315 of FIGS. 3A and 3B operates in a slightly different manner. In this embodiment the barrier 317 of the valve 315 comprises an aperture 319 that allows an airflow to pass through the barrier 317. Thus, in this embodiment, in the first position (FIG. 3B), the valve 315 extends across the first passage 306 and an airflow passes through the aperture 319. In the third position (FIG. 3A) the valve 317 is retracted from the passage 306, but the airflow is blocked by a shroud 320 of the biasing member (i.e., a compression spring 318). Thus, in this embodiment the valve 315 is biased by the compression spring 318 into the third position.

In the embodiment of FIGS. 4A and 4B, the valve 415 is hingeably mounted, by way of a hinge 421, to a wall of the first passage 406. Thus, the valve 415 is configured to rotate (about the hinge 421) between the first (FIG. 4A) and second (FIG. 4B) positions. In particular, the valve 415 rotates in response to an airflow in the first passage 406. This rotation is facilitated by lip 422 at a distal end of the barrier 417. As is shown in FIG. 4B, rotation of the valve 415 causes the barrier 417 to partially obstruct the first passage 406. In this embodiment the valve 415 is biased towards the first position by a biasing member 418 extending between the valve 415 and the wall of the first passage 406 to which the valve 415 is mounted.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilized for realizing the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the invention that may provide certain benefits under some circumstances. It is to be appreciated, however, that other embodiments may also be preferred under the same or different circumstances. The recitation of one or more preferred embodiments therefore does not mean or imply that other embodiments are not useful, and is not intended to exclude other embodiments from the scope of the disclosure, or from the scope of the claims.

1.-131. (canceled)

132. A smoking substitute apparatus comprising:
a first passage for receipt of an aerosol flow;
a second passage arranged in parallel with the first passage and comprising an additive delivery portion for delivering an additive to an airflow in the second passage;
a mouthpiece comprising an outlet in fluid communication with the first and second passages; and
a valve for controlling aerosol flow through the first passage, the valve movable between:
a first position in which an airflow drawn through the outlet is received substantially exclusively from first passage; and
a second position in which an airflow drawn through the outlet is received from a combination of the first and second passages.

133. A smoking substitute apparatus according to claim 132 wherein, in the first position, the first passage is substantially unobstructed by the valve.

134. A smoking substitute apparatus according to claim 132 wherein, in the first and second positions the second passage is substantially unobstructed by the valve.

135. A smoking substitute apparatus according to claim 132 wherein in the second position the first passage is at least partially obstructed by the valve.

136. A smoking substitute apparatus according to claim 132 wherein the valve is movable to a third position in which an airflow drawn through the outlet is received substantially exclusively from the second passage.

137. A smoking substitute apparatus according to claim 136 wherein, in the third position, the valve substantially obstructs the first passage.

138. A smoking substitute apparatus according to claim 132 wherein the second passage comprises an airflow restrictor for restricting airflow through the second passage.

139. A smoking substitute apparatus according to claim 132 wherein the additive delivery portion is configured to restrict airflow through the second passage.

140. A smoking substitute apparatus according to claim 137 wherein the additive delivery portion comprises a porous medium.

141. A smoking substitute apparatus according to claim 132 wherein the valve is biased into the first position.

142. A smoking substitute apparatus according to claim 132 wherein movement of the valve between the first and second positions is in a direction generally transverse to a longitudinal axis of the first passage.

143. A smoking substitute apparatus according to claim 132 wherein the valve is slidably engaged with a wall of the passage.

144. A smoking substitute apparatus according to claim 143 wherein the valve comprises a pushbutton for a user to move the valve between the first and second positions.

145. A smoking substitute apparatus according to claim 132 wherein the valve is configured to rotate between the first and second positions.

146. A smoking substitute apparatus according to claim 132 wherein the valve is configured to move from the first position to the second position in response to a pressure drop in the first passage.

147. A smoking substitute apparatus according to claim 132 wherein the first passage comprises an aerosol generator for delivering an aerosol to an airflow in the first passage.

148. A smoking substitute apparatus according to claim 147 wherein the valve is between the aerosol generator and the outlet.

149.-312. (canceled)

313. A smoking substitute apparatus according to claim 134, wherein the second passage has a greater resistance to airflow than the first passage.
